(19) United States
(12) Patent Application Publication
(10) Pub. No.: US 2021/0005303 A1
(43) Pub. Date: Jan. 7, 2021

(54) PATIENT CARE SYSTEM

(71) Applicant: ARES TRADING S.A., Aubonne (CH)

(72) Inventors: Laurent MERCIER, Bernex Geneva (CH); Ivan VECERINA, Lausanne (CH); John MULCAHY, Nyon (CH); Kouvas GEORGIOS, Geneva (CH)

(21) Appl. No.: 16/754,855

(22) PCT Filed: Oct. 15, 2018
(86) PCT No.: PCT/EP2018/078084

§ 371 Date: Apr. 9, 2020

(30) Foreign Application Priority Data
Oct. 13, 2017 (EP) 17196431.5

(51) Int. Cl. G16H 20/30; G16H 10/20; G16H 15/00; G16H 80/00

(52) U.S. Cl. CPC G16H 20/30; G16H 80/00; G16H 15/00; G16H 10/20

(57) ABSTRACT

There is disclosed an interactive patient care system configured to provide care to a patient suffering from a chronic disease or condition, comprising a server system (6) configured to receive and transmit data via a communication network (16) to and from users including 5 patients (15a) and health care professionals (15b). The server system comprises a processing unit (6e), a memory (6f), a patient database (6c) configured to store data related to the patient and a library database (6d) configured to store data related to predefined therapeutic interventions obtained on evidence-based care pathways. The server system further comprises an application server (6b) and a communication server (6a) including a web server 10 software application for data transfer through the internet. The application server (6b) comprises a patient care system software for chronic disease or condition management, configured to: i) select for a given patient at least one predefined therapeutic intervention from the library database based at least on the data related to the patient stored in the patient database, and ii) generate a personalized care plan based at least on the selected predefined 15 therapeutic intervention. The patient care system software comprises patient therapy services (40) comprising health monitoring components (40c) configured to monitor health parameters and/or treatment adherence parameters of the patient. The therapy services are configured to provide health care assistance to the patients based on said personalized care plan.

### FIELD OF THE INVENTION

The present invention relates to an interactive patient care system configured to provide care to a patient suffering from a chronic disease or condition such as for example Multiple Sclerosis, Rheumatoid Arthritis or chronic heart failure. More particularly, the patient care system is indicated for use by patients and healthcare teams for tracking and reviewing patient data in order to support the long-term management of chronic disease and condition.

### BACKGROUND

Healthcare systems and healthcare providers are experiencing increased financial pressure due to a combination of patient demographics, the burden of chronic disease management and constrained budgets. Moreover, the current healthcare system is experienced by patients as disjointed, made of isolated encounters with individual health care professional that focused on separate aspects of their care. Current care is delivered by schedule with fixed and often distant periodic appointments with specialists failing to timely capture and address treatments needs. Patients are therefore not optimally treated between consultations, as available patient support programs and other local services often rely on incomplete or outdated information. In addition, care intervention lack coordination, frequently resulting in duplicated, redundant, and even conflicting interventions.

WO2016/151364 discloses an electronic patient care system comprising a medical device for administering a medical treatment to a patient and a server system configured to receive and transmit data via a communications network to, respectively from users including patients and health care professionals. The server system is further configured to process and store data related to patient care. In this respect, the server system comprises a database configured to encrypt and store encrypted data related to patient care, an application server including patient care software components for disease management and patient information management and a communication server including a web server software application for data transfer through the internet. The patient care software components are operable to receive medical device usage data comprising data on the usage of said medical device transferred through the communications network, and further operable to process said medical device usage data in conjunction with patient data to generate a report or a plurality of reports related to the treatment of the patient.

This system is particularly configured to improve patient treatment monitoring for health care professionals to gain a better understanding of the effects of the treatment regimen on the health of the patient where the effects of drug administration have a large temporal offset from the time administration for the treatment of many chronic diseases. However, this system is mainly designed for monitoring patient's treatment with a specific drug, in particular to monitor adherence of drug administration for chronic diseases that have been diagnosed.

There is therefore a need for a digital health solution that improves care coordination by enabling the deployment of personalized, integrated care plans, offering a cohesive source of information on a patient's condition and treatment in a way that the patients perceive a continuity and coherence in their interactions with the entire team of care professionals.

An object of the invention is therefore to provide an interactive patient care system supporting a data-driven decision making process with updated information made available when and where needed in order to improve the care provided between consultations.

It is advantageous to provide a patient care system that delivers chronic care that targets one or more chronic conditions, allowing HCPs to familiarize themselves for a single system that can accommodate a broad range of patients, and that is both localized and personalized.

It is advantageous to provide a patient care system that provides easy and effective health communication between HCPs and patients.

It is advantageous to provide a patient care system to provide remote patient monitoring and medication adherence tracking across a wide range of therapies, including medications but also digital health interventions.

It is advantageous to provide a patient care system that integrates nudges, messages and educational materials that help patients adopt an improved lifestyle and behaviour.

### SUMMARY OF THE INVENTION

Objects of the invention have been achieved by the system and method according to the independent claims. Various advantageous features of the invention are set forth in the dependent claims.

Disclosed herein is an interactive patient care system configured to provide care to a patient suffering from a chronic disease or condition. The patient care system comprises a server system configured to receive and transmit data via a communication network to and from users including patients and health care professionals. The server system comprises a processing unit, a memory, a patient database configured to store data related to the patient, including parameters of their personalized care plans, and a library database configured to store data related to predefined therapeutic interventions obtained on evidence-based care pathways. The server system further comprises an application server and a communication server including a web server software application for data transfer through the internet. The application server comprises patient care system software for chronic disease or condition management, configured to: i) select for a given patient at least one predefined therapeutic intervention from the library database based at least on the data related to the patient stored in the patient database, and ii) generate a personalized care plan based at least on the selected predefined therapeutic intervention. The patient care system software comprises patient therapy services which include health monitoring components configured to monitor health parameters and/or treatment adherence parameters of the patient. Therapy services are configured to provide health care assistance to the patients based on said personalized care plan.

In an embodiment, the personalized care plan is generated based on one or more additional parameters selected from the group consisting of patient's goals, patient's preferences, patient's schedules, input provided by the patient and patient's historical monitored data.

In an embodiment, the patient care system software comprises a patient information management program with includes a reports component configured to generate reports, in the form of tables, charts, lists, diagrams or graphical representations based on information selected from drug administration history, adherence data, patient outcome reports, patient health reports, patient physiological data reports, medical device settings, treatment regimen data, and any combination of aforesaid information.

In an embodiment, the reports component are configured to form composite reports for simultaneous display on a user interface device (UID) display, including composite adherence and patient outcome reports to facilitate evaluation of the effects of non-adherence to the treatment regimen or the efficacy of the medical treatment.

In an embodiment, the patient therapy services include tests accessible online by patients. The monitor components are configured to capture results of tests, such as for example vision, agility or walking tests.

In an embodiment, the patient therapy services include digital health interventions, such as training exercises accessible online by patients, including training exercises selected from cognition training and mobility training.

In an embodiment, the patient therapy services may further be configured to receive data from sensors relating to physiological measurements of the patient's measured physiological data captured automatically by sensors and transmitted to the server system via the patient's user interface device and/or medical device and/or by the sensing or training device, physiological data selected from body temperature, blood pressure, pulse rate, electroencephalography measurements, electrocardiography measurements, breathing sensor measurements, blood sugar sensor measurements.

In an embodiment, the patient therapy services comprise web-based software which resides on the application server.

In an embodiment, the patient therapy services comprise software residing on a patient's user interface device.

In an embodiment, the communication server further comprises a remote service data upload software application configured for wireless telecommunication technology (WTT) data transfer.

In an embodiment, the patient care system further comprises a client side software application installable on a personal computer or on a mobile user interface device (UID), such as a phone or computer tablet, configured to upload medical device usage data to a patient information management program application on the server system.

In an embodiment, the patient care system software further comprises prioritization and care coordination management configured to allow health care professional to review monitored patients, in a prioritized order, when monitored heath parameters present deviations or anomalies that exceed at least one predetermined threshold value.

In an embodiment, the prioritization and care coordination management are configured to generate different levels of signals based on the severity of said deviations or anomalies. The signal level is used to prioritize patients for care assistance. Prioritization and care coordination management may optionally be connected to a call centre.

In an embodiment, the patient care system software further comprises a notification services software component configured to transmit notifications by email, SMS (Short Message Service) and/or by using an application operating on a mobile device to patients and optionally other users of the system.

In an embodiment, the notifications are selected from the group consisting of encouragement, tip, request for patient to complete an activity and questions to be answered by the patient.

In an embodiment, the notification services software component is configured to send messages to the patients based on one or more types of parameters selected from the group consisting of monitored heath parameters, monitored treatment adherence parameters and target parameters.

In an embodiment, the target parameters are set in the personalized care plan of the patient.

Also disclosed herein is a method of monitoring treatment and providing care to a patient suffering from a chronic disease or condition. The method comprises:

providing an interactive patient care system comprising a server system configured to receive and transmit data via a communications network to and from users including patients and health care professionals, the server system comprising a patient database configured to store data related to the patient, a library database configured to store data related to predefined therapeutic interventions obtained on evidence-based care pathway, an application server including a patient care system software for chronic disease or condition management, and a communication server for data transfer through the communications network;

processing and storing data related to predefined therapeutic interventions obtained on evidence-based care pathway on the server system; processing and storing data related to patient care on the server system;

selecting at least one predefined therapeutic intervention based at least on the data related to the patient care;

generating a care plan template based at least on the selected predefined therapeutic intervention;

creating a personalized care plan for the patient based at least on the care plan template, and

providing patient therapy services to assist the patient to follow his personalized care plan.

In an embodiment, the patient therapy services comprise one or more wellness services selected from the group consisting of coaching services, encouragement for behaviour change and online exercises.

In an embodiment, the patient therapy services comprise e-Health therapies which includes medical devices designed to assess or contribute to the care of a specific chronic disease or condition.

In an embodiment, the method further comprises providing a client side software application accessible on a personal computer or on a mobile user interface device (UID), such as a phone or computer tablet, configured to upload medical device usage data to a patient information management program application via a remote service data upload application on the server system.

### BRIEF DESCRIPTION OF THE DRAWINGS

The invention will be better understood with the aid of the description of embodiments given by way of example and illustrated by the figures, in which:

FIG. 1 is a schematic overview illustration of a patient care system according to an embodiment of the invention;

FIG. 2 is another schematic overview illustration of a functional architecture of a patient care system according to an embodiment of the invention.

FIG. 3 is a schematic overview illustration of a functional architecture of a server system of a patient care system according to an embodiment of the invention;

FIG. 4 is a schematic overview illustration of a software system context diagram of a server system of a patient care system according to an embodiment of the invention; and

FIG. 5 is a schematic overview chart of software components of a patient care system according to an embodiment of the invention;

### DETAILED DESCRIPTION OF EXEMPLARY EMBODIMENTS

In the following description, in exemplary embodiments of the present invention the patient care system and method for providing care to a patient are configured for treatment of chronic diseases or conditions such as Multiple Sclerosis, Rheumatoid Arthritis or chronic heart failure.

The present invention may also be applied in treatments for other diseases such as, juvenile rheumatoid arthritis, psoriasis, plaque psoriasis, Crohn's disease, juvenile Crohn's disease, asthma, psoriatic arthritis, ulcerative colitis, systemic lupus erythematosus, ankylosing spondylitis, chronic obstructive pulmonary disease, and diabetes.

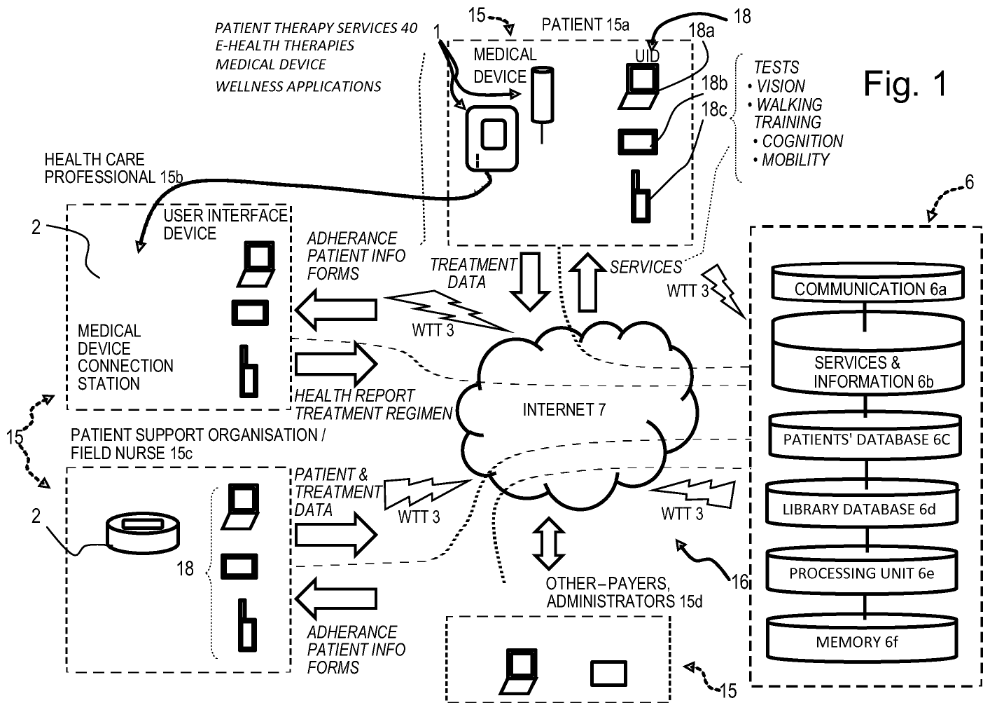

Referring to FIG. 1, a patient care system for providing care to a patient suffering from a chronic disease or condition according the invention comprises a server system 6, a user interface device 18 and patient therapy services 40 which comprises, depending on the embodiments, software and optionally hardware such as a medical device. The server system 6 is configured to process and/or store information related to patient care and accessory services, such information being received and transmitted via a communications network 16, in particular a global computer network such as the internet 7. The communications network may further include a wireless telecommunications transfer (WTT) network, for instance a mobile phone network 3, and direct point-to-point communication between users 15 and the server system 6.

Users include patients and health care professionals (HCP), in particular physicians and nurses. Users may further include patient support organisations or persons (PSO), for instance including care coordinators, field nurses, or informal caregivers such as relatives of the patient. Users may further include one or more of system administrators, health insurance organisations, suppliers of health services, manufacturers and suppliers of medication, pharmacies, and payers of health services. HCPs manage the patient related information in the system. This role creates patient entries, and manages and monitors the patients' related data in the system to enable better treatment and care to the patients. A system administrator is responsible for managing the system related functions in the applications that includes creating users, roles and functionalities, and other master data. The role of a care coordinator may be similar to that of the HCP and can manage all patient data. The care coordinator may typically access the application using a mobile user interface device, such as a computer tablet or smartphone. A care coordinator may for instance schedule appointments, meet the patients, and collect information from the patient. An informal caregiver may have only read-only access to information about a patient they are concerned to, allowing them to provide support and encouragement as needed.

User interface devices 18 may comprise a display and means to communicate with the server system 6 and may include personal computers 18a, computer tablets 18b, mobile phone devices 18c or other electronic computing devices with a graphical user interface and means to communicate with the server system 6.

The data are stored confidentially in the server system 6 and processed and classified so as to be easily and securely accessible to the various users 15a, 15b, 15c, 15d as a function of the access rights allocated to each type of user and the identity of the user. Medical information on the patient is accessible to the patient 15a and the health care professional 15b, possibly also to authorized members of the patient support organisation 15c, such as care coordinator, but not to other users.

The server system 6 may comprise a plurality of physical and/or virtualised servers in a single location or in a plurality of locations distributed in the communications network 8. The meaning of “server” thus encompasses hardware servers with server software dependant on the hardware server configuration, and virtualised servers installed on one or more hardware server components that are independent of the hardware server configuration. A server may also be formed by a plurality of server hardware and/or software components that are distributed in a computer network.

Still referring to FIG. 1, the server system 6 comprises a communications server system 6a including a web server (HTTP server), an application server 6b, a patient's database 6c configured to store data related to patients including health conditions, a library database 6d configured to store data related to predefined therapeutic interventions, a processing unit 6e and memory 6f. The predefined therapeutic interventions are built on evidence-based care pathways and adapted to the local healthcare system thereby delivering chronic care that targets multiple specialty chronic conditions in a drug-agnostic manner. Predefined therapeutic interventions are based from various updated sources. The patient care system therefore provides personalized patient care at home on an as needed basis, and addresses the unmet need for fully integrated patient care with personalized home-based care that adheres to evidence-based and locally adapted clinical pathways

The application server 6b comprises a patient care system software configured:

to select for a given patient at least one predefined therapeutic intervention from the library database based at least on the data related to the patient stored in the patient database, and to generate a personalized care plan based at least on the selected predefined therapeutic intervention.

The patient care system software also comprises patient therapy services 40 which include health monitoring components, such as medical device 1, configured to monitor health parameters and/or treatment adherence parameters of the patient in order to provide health care assistance to the patient based on personalized care plan and monitored heath parameters and/or monitored treatment adherence parameters.

The application server system 6b may comprise an information server for handling online services accessible by the users, a notification server for handling notification services to users, a server for handling mobile components, and a server for handling data load services from local components and devices.

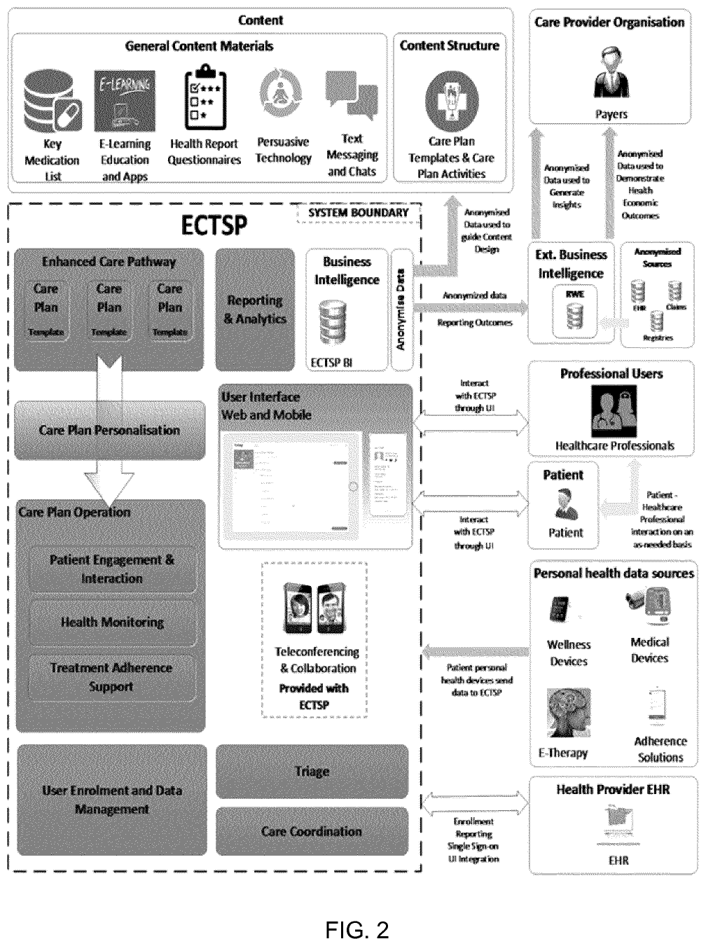

FIG. 2 shows a schematic overview illustration of a functional architecture of a patient care system integrating an e-Health Chronic Treatment Solution Platform (ECTSP) previously and hereafter referred to as the patient care system software (for chronic disease or condition management). The key functional requirement groups that make up the patient care system software comprise:

### Care Plan Management

Care plan management provides personalized care plans which are central to offering a coordinated care experience, addressing the market needs. By providing care plan templates and a library of defined therapeutic interventions, the patient care system software allows the HCP to create a comprehensive personalized care plan in a few minutes.

### Care Plan Operation

Digital Health Interventions (E-Therapies and Mobile Health) The patient care system software supports the delivery of e-Health Therapies and wellness applications. E-Therapies are validated medical devices designed to assess or contribute to the care of specific conditions. Wellness applications are devices that encourage activities which improve general wellness or behavior changes. E-Therapies include physical rehabilitation, cognitive training, and other computer-based training solutions (e.g. for depression management). Wellness applications may include a variety of applications such as providing coaching, encouragement for behavior change, or online exercises
Patient Engagement & interaction

Patients who are engaged in the management of their chronic disease will achieve better outcomes. The patient care system software helps activate patients and involve them in their own care. This requires careful integration and coordination of reminders, queries, and prompts sent to patients, in order for the patient care system software to be perceived as useful and unobtrusive. The patient care system software supports interactive exchanges with patients, which can be driven by the following inputs: a patient's personalized care plan, including goals, schedules, and targets defined therein; a patient's preferences and demographics stored in a patient's profile; signals originating from automated patient monitoring; patient's level of activity and usage of a software application; and previous interactions, responses, inputs provided by the patient.

Scripted interactions may deliver the following to patient: encouragements or tips; requests for the patient to complete an activity (including e-Therapies, or the completion of a Health Report Questionnaire) and (Multiple-choice) questions to be answered by the patient. Examples of scripted interactions may include: a sequence of hints and messages that are carefully crafted to help the Patient and to encourage an improvement of his/her lifestyle; encouragements or hints based on the patient's performance in achieving an objective defined in a personalized care plan; and queries focusing on collecting trending information on key symptoms experienced by the patient. These scripted interactions may be presented in the form of interactive chats, where messages received patients are predefined (“scripted”), and patient provide a response either through direct selection from a predefined set of allowed answers, or using other input methods (voice, text input) that are interpreted by the system to select the next message to be given.

### Monitoring Integration

Based on the personalized care plan, the patient care system software automatically collects information from a variety of sources, allowing to track and trend aspects of a patient's evolution. Monitored parameters include: the outcome or performance of the therapeutic interventions defined in a care plan; adherence to oral or injectable medications, to planned e-Therapies or exercises, and to assimilating and following educational materials; patient reported outcomes collected using health report questionnaires and other queries or instruments; information recorded by medical or consumer devices, such as blood pressure and activity trackers; and any other data that is recorded in relation with a patient.

### Triage and Care Coordination

Triage refers to a service that allows HCPs to review, in a prioritized fashion, monitored patients for which deviations or anomalies have been observed. The patient care system software provides a dedicated triage interface which allows monitoring of local populations of patients according to a number of monitored parameters. The triage interface supports continuous monitoring of patients and provides views and reports that allow triage users (e.g. HCP or care coordinator) to inspect individual patient data, or to review specific lists of patients. The triage interface distinguishes signals of specific monitored parameters and allows triage users to make targeted interventions.

One of the action types associated with a monitoring rule can raise a triage signal when the monitoring rule is met. Some rules can be set up so that a positive message is sent automatically (e.g. “Congratulations, you have met all of your health goals in your personal care plan over the last 20 days”); other rules can trigger the involvement of a call center (e.g. if adherence drops below 80% for 2 weeks running then the triage service is notified). Triage signals, triggered by a rule, are categorized by level of severity (missing data, minor, moderate, major), and remain active and associated with the patient until the signal is addressed. Active signals can be reviewed by a triage user, or may automatically trigger the sending of a nudge to the patient. Triage views allow personnel from patient support services or call centers to review the signals associated with all the patients being monitored.

During the creation of a care plan, or upon request of an HCP or patient, or as a triage intervention for a signal, an appointment can be created in the patient care system software. When an external scheduling system is available, the patient care system software could generate and confirm an appointment automatically. In the absence of such a system, a task could be automatically created for a nurse or other technical or administrative personnel to schedule an appointment. Scheduled patients appointments are visible in order to generate reminders to patients, and as a viewable information that supports care coordination.

### User Enrolment and Data Management

Access to the personal information of each patient is restricted to identified personnel connected with that patient. During the enrolment process, a patient's informed consent must be recorded in written or electronic form. Subsequently, every system user needs to accept terms and conditions, to access individual data, which can be recorded electronically. To enable consent traceability the system delivers the means to maintain consent management.

### Reporting and Analytics

The patient care system software generates a variety of reports, based on a broad range of information that it collects on the usage of the system, on the recording of remote interventions, and on the usage and performance of care provided to patients. This allows the system to provide reports that support continuous improvement of provided care. The collection of real world evidence also supports the evaluation of medical and heath economic outcomes.

The patient care system software is designed to collect data on treatment performance, with the intent to continuously optimize care efficacy, efficiency and treatment outcomes. Collected information may be combined with external information sources, and predictive analytics will be applied in order to generate actionable insights. The patient care system software is configured such that it allows for capturing, storing and analyzing relevant and actionable data metrics. Such data may be utilized to determine insights and achieve better outcomes in the context of delivery of new, digitized care pathways. Analytics also enable patients to gain insights by anonymously comparing their journey with those experiencing similar conditions. Data may include, but is not limited to, platform usage information, relevant clinical data and signals processed and generated by the system, as well as appropriate non-clinical data. Data captured, stored and analyzed adhere to relevant privacy and security standards including working with all involved stakeholders to address data ownership and anonymization concerns. Also physicians and payers will gain insights on the group of patients or the group of care givers they have under management, without compromising individual patients interests and privacy.

Outside of the patient care system software boundaries are external systems and actors that supplement the patient care system software as defined below:
Clinical content which defines the configuration of care pathways supported by the patient care system software
General system content which defines:
   Medications
   E-Learning and e-Therapies, including education materials and software applications, such as digital health interventions
   Text messaging and chats, including:
      Messages such as behavioural nudges, reminders and notifications
      Scripted interactions (interactive chats)
Local services which provide integration to:
   Electronic Health Records (EHRs)
   Billing systems
   E-prescription systems
   Prior-authorisation systems
   Remote consultation systems
Monitored sources which provide outcomes data from:
   Medical devices or physiological monitoring devices
   Digital Health interventions such as E-Therapy, or
   Wellness devices
Business intelligence component which integrates pseudonymized data collected in the patient care system software with other anonymized date sources in order to generate medical insights and assess heath economic outcomes. Business intelligence also guides the further improvement of content materials.

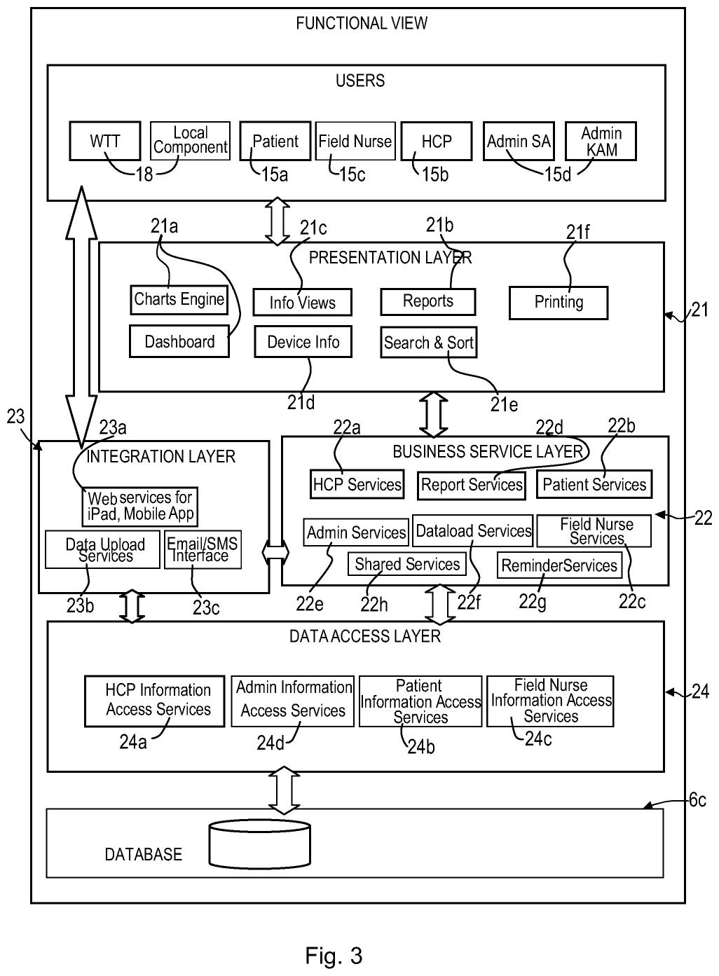

Referring now to FIG. 3, a functional diagram of the program architecture of an exemplary embodiment of a computerized patient care system according to the invention is illustrated. The program functions comprise presentation program components 21, business services program components 22, integration program components 23, and data access program components 24.

The presentation components are responsible for managing the requests and response from the user's interface device, for instance from a network browser on the user's interface device, and rendering the presentation to the respective users. The presentation components are specialized to render charts 21a, reports 21b, views 21c, device information 21d, data entry and search 21e, and prints 21f.

The business services components are responsible for providing all the functionality required for the HCP and administrator users. They hold the business logic to process and provide the data back and forth to the users. The business services components are grouped based on the functionality they provide to the users, and include HCP services 22a, patient services 22b, care coordinator services 22c, report generation services 22d, administrative services 22e, data load services 22f, reminder services 22g, and shared services 22h. The shared services provide functionality, such as logging, error handling, audit trail, caching, security, and notification used by all the other program modules.

Integration components are responsible for providing the interfaces to various external interface devices, including web services for mobile applications 23a, data upload services 23b, email and SMS interface 23c. These program components may include: programs to upload device data from the local component and the WTT system, Web-services to provide patient and associated data to mobile applications (e.g. iPad™ application), Web-services to provide care coordinator and associated data to mobile applications (e.g. iPad™ application), E-mail gateway integration and SMS gateway integration.

The data access components provide functionality to manage data storage and retrieval from the database, and include HCP information access services 24a, patient information access services 24b, care coordinator information access services 24c and administrator information access services 24d.

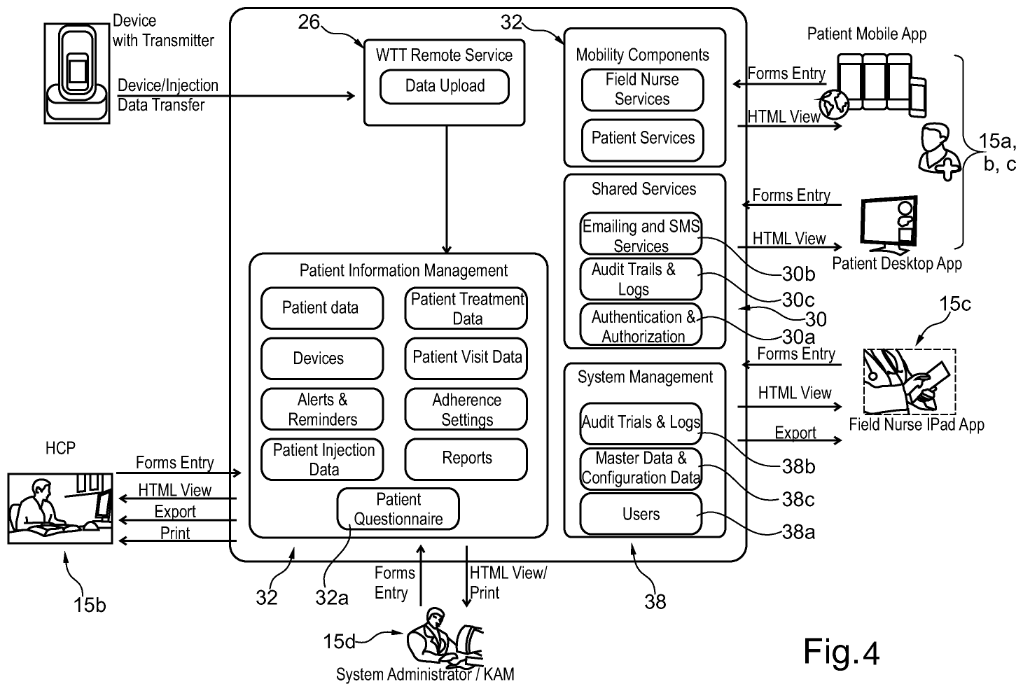

FIG. 4 illustrates an exemplary embodiment of the interactions between users and the server system on a contextual level. In this example, the various users, including patients 15a, health care professionals 15b, care coordinators 15c, and administrators and key account managers 15d access the software applications on the server system 6 remotely through the communications network 8, including in particular through the internet 7 (FIG. 1). Administrator, account managers and health care professional may typically access the system via a user interface device (UID) 18 in the form of a desktop computer for instance, whereas patients and care coordinators may typically access the system through a desktop UID as well as a mobile UID in the form of a smart phone or computer tablet.

For the mobile UID's, medical device data may be uploaded into the server system 6 using a WTT remote service data upload application 26 in the server system, directly from the medical device through the wireless telecommunications network 10 using the available mobile cellular systems, for instance GPRS (general packet radio service), UMTS (Universal Mobile Telecommunications System) or others. UlDs accessing the server system through the internet 7 can also upload medical device data, in particular usage data stored in the medical device.

Only registered users can access the server system. Users are authenticated by an authentication and authorization program application 30a forming part of a shared services section 30 available to all users. User data, including identity, type, and respective roles and privileges is stored in the server system, for instance in the database 6c, and enables users to have access to authorized functionality and data based on their respective roles and privileges, for instance patient sensitive data may be available only to HCPs and patients, whereas server system administration functions may be available only to system administrators, and forms configurations available only to key account managers and system administrators.

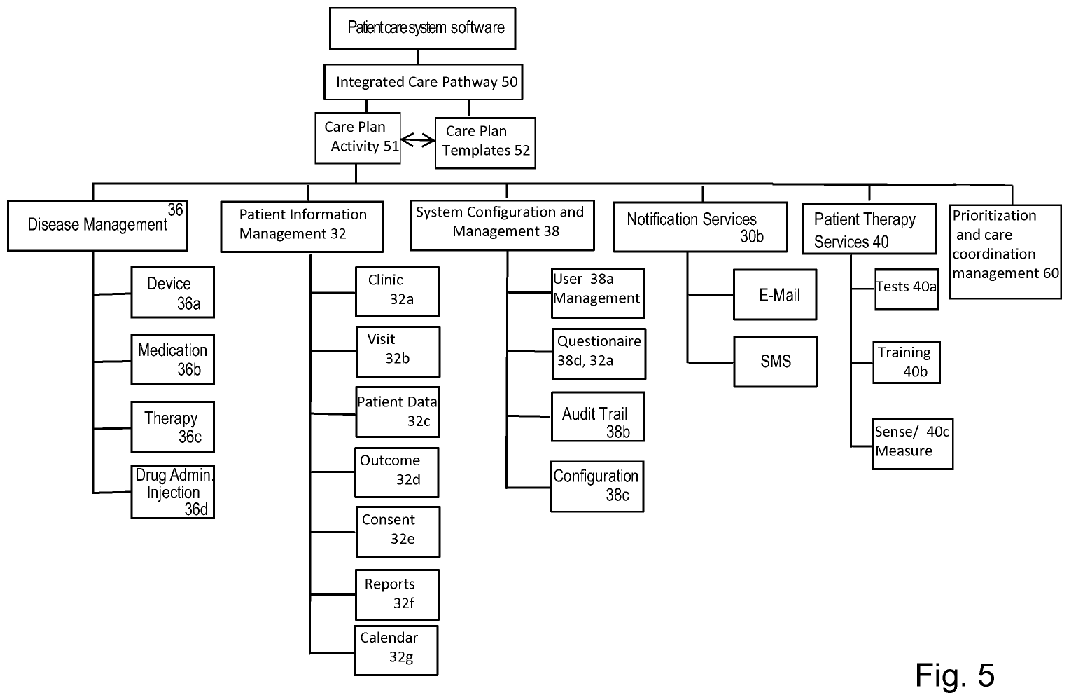

Referring to FIG. 5, in conjunction with FIG. 4, software components of the patient care system software according to the invention may be categorized as follows:

i. Integrated Care Pathway 50

Integrated care pathways support a holistic and integrated care of patients. Configuration profiles for treatment intervention allow them to automatically be associated with adherence monitoring, nudges and reminders, patient education materials, side effect monitoring, or other predefined interventions. Treatment interventions may or may not be associated with a drug. Support for care pathways is implementing using two key concepts, the Care Plan Activity and the Care Plan Template as defined hereafter.

ii. Care Plan Activities 51

Care Plan Activities (CPA) are therapeutic interventions which can be independently selected and associated with a patient. Each CPA integrates the definition of a treatment, education materials, messages (nudges, reminders), monitoring needs and configuration settings.

iii. Care Plan Templates 52

Care Plan Templates (CPT) accelerate the creation of personalized care plans by providing a set of treatment options appropriate for a given category of patient. The CPT identifies a list of frequent needs, problems, symptoms or comorbidities associated with a patient category, and offers a selection of possible CPA to choose from in order to address each problem. For example, a CPT for a patient can serve to prompt the referring HCP to identify:

a medication for disease activity control: Humira®, Orencia® (each is implemented by a dedicated CPA.

an education package on “Living with Rheumatoid Arthritis”

activity management: a choice between the use of a monitoring device, rehabilitation using patient therapy services or referral to an occupational therapist.

Templates may include information on the characteristics of patients to which they apply (key diagnostic, demographics), allowing appropriate templates to be presented to the refereeing health care professional for a given patient. Some templates may allow a lot of flexibility and addition of any CPA. Other may be more restrictive as may be desired in some care provider organizations.

iv. Disease Management 36 This category comprises software components which are used to manage the disease. These are software components that relate to the medical device configuration and use 36a, medication 36b, therapy 36c and drug administration 36d , e.g. injection.

v. Patient Information Management 32 This category comprises software components which are used to manage information on patients. These include information related to the treating clinic 32a, medical and therapeutic visits 32b, patient consents 32e, patient data 32c, treatment outcomes 32d, patient and HCP reports 32f, and calendar 32g software components.

vi. Notification Services 30b

This category comprises software components which are used for notification to the patients to ensure their engagement in the management of their chronic disease in order to achieve better outcomes. These include e-mail and SMS software components 30b that provide patients with hints, reminders, feedback and tips that helps patient achieve their treatment objectives. When appropriate, the patient is asked for information that complements automated monitoring, which support efficient involvement of the health care professionals.

vii. Patient Therapy Services 40 This category comprises software components which are used to provide information to the patient inter alia to assist in improving therapy, disease management and patient reports. Patient therapy services software components include online tests 40a, such as vision and walking tests, online training 40b such as cognition and mobility training, disease information for patients and patient support organisations, and online patient physiological monitoring.

viii. Prioritisation and coordination management 60

This category comprises software components which are used to allow HCPs to review, in a prioritized fashion, monitored patients for which deviations or anomalies have been observed. The patient care system is configured to generate signals as a result monitored deviations or anomalies. Different levels of signal can be generated by the system based on the level of importance and urgency. For instance, a low level signal may be generated when a monitored parameter of the patient exceeds a low level threshold value while a high level signal is generated when the monitored parameter exceeds a high level threshold which is indicative of an emergency situation. The system can therefore prioritize patients which need immediate intervention over patients whose monitored deviations or anomalies do not represent a life-threatening situation. The patient care system makes all information it manages available to the patient and each member of the patient's care team. Individual patient summary views are available prior to a visit. Information gathered during visits or calls can be recorded. Through collaboration, the patient care system facilitates and leverages the involvement of caregivers such as family members to provide additional support and encouragement to the patient.

Depending on the type of disease to be treated, additional patient therapy services may include rehabilitation, stress monitoring, cognitive behavioural therapy, depression assessment and/or treatment, fatigue monitoring and treatment, activity tracking and gait assessment. Stress monitoring may for instance include measurements of heart rate and oxygen in the blood. Depression may be assessed and/or treated by the present system and device as a secondary disease in conjunction with e.g. multiple sclerosis, or as a primary disease.

Patient therapy services may further include alarms, for instance alarms for administration of medication and alarms for visits to the treating clinic, the HCP, or the care coordinator. The online capture of results of tests may be fed into the reports 32f component and the performance of training may also be recorded and available to the reports component or as information available to the HCP and filed nurse. Alarms may be sent by the notification services component 30b. Tests may be done by the patient at home using the patient's UID 18 and test apparatuses such as motion sensors connected to the UID 18 by a wired or wireless connection. The tests may be vision tests, walk tests, cognition tests and/or mobility tests. Patient physiological monitoring may also include physiological measurements of the patient's measured physiological data, such as body temperature, blood pressure, pulse rate, height (in the case of a growth hormone treatment) of the patient. Certain physiological measurements may be captured automatically by sensors and transmitted to the server system via the patient's UID 18 and/or medical device 1 an/or by the sensing or training device if it is equipped with communications means for data transfer over the internet 7 or the WTT network 3. Certain physiological data may be manually entered by the patient 15a or the HCP 15b or the care coordinator 15c.

In an exemplary embodiment, the software components illustrated in FIGS. 3 and 4 and presented above may comprise the following features:
Therapy 36c—this software component provides functionality related to drug therapy which uses medication to treat the disease. Therapy is associated with medication as medication is governed by a Therapy.
Medication 36b—this software component provide functionality related to medication prescribed or taken by the Patient. Medication component is associated with Therapy as Therapy governs the Medication given to patients.
Device 36a—this software component provides functionality related to the medical device 1.
Device component 36a stores medical device usage data, for instance injection data, and is also associated with patient data 32b as every patient will have at least one device associated with him/her.
Injection 36d—this software component provides functionality to store the date and time of drug administration, for instance injections, and their related information (e.g. injected dose). Injection data will be passed to Device component 36a.
Patient Data 32c—this software component provides maintenance of patient data which is gathered using Device component 36a and a web application.

Clinic 32a—this software component manages the association of a treating clinic to a patient 15a.
Consent 32e—this software component manages the consent of the patient 15a for being treated by a primary physician or other HCPs 15b whoever might be involved in treating the patient in the same clinic where patient is undergoing the treatment.
Patient Outcome 32d—this software component will provide the outcome of questionnaire presented to patient and will use Report component 32f for generating the health reports for a patient 15a.
Patient Visit 32b—This software component manages the visit information of the patient entered by the HCP 15c and care coordinator 15d.
Reports 32f—this software component generates patient reports from the data received through medical device usage received from the medical device that provides adherence data, questionnaires filled by the patient that may provide patient reported outcomes, adherence settings including information on the treatment regimen, and visit data included by the HCP and care coordinator. Reports advantageously include composite diagrams that present in a single view multiple reports along a common time scale, including patient reported outcomes (PRO) and adherence information along a common time scale. The afore mentioned composite diagram enables the HCP to more easily and quickly assess the effects of non-adherence of a patient to the prescribed treatment regime and if needed to propose corrective measures such as reminding the patient on importance of adherence, enhanced monitoring of adherence, increased frequency of visits, a modification in the treatment regime, and additional treatment or therapeutic measures.
Calendar 32g—this software component will manage calendars for HCPs and patients for providing details on events as well as scheduled visits. Calendar software component 32g may also issue reminders of an event to patient or HCP.
Questionnaire 38d—this software component manages all the questionnaires presented to the patient and their responses. This feature may provide outcomes of surveys provided by the patient information management module 32 to the patients. The questionnaires include clinically validated questionnaires directed to the patient, the responses of whom are used by the Reports component 32f to generate patient reported outcome (PRO) reports.

Certain of the software components described herein may be in the form of client-server applications whereby a client side application is installed on the user interface device (UID) to allow the UID to connect to the application on the server system and run certain features locally. Certain software components described herein may be in the form of browser applications that are accessed and run through a web browser on the users UID. Certain software components may also be installed and run independently on various devices of the system including medical devices, medical device connection stations, and user interface devices, depending on the type and function of the software application. The use of the term “software component” herein may mean a portion of a program, a program, a plurality of portions of programs or a plurality of programs that work together to achieve the desired function.

The integration of the aforementioned software components in the server system 6 communicating with client applications installed on both mobile and wired user interface devices 18 described herein provides a flexible global patient care system optimising the quality of information available to patients, HCPs, care coordinators and other members of patient support organisations as well as other actors such as drug manufacturers to improve overall patient care and health, while reducing the healthcare costs. Moreover, over time, data accumulated in the database allows the HCPs and drug providers to gain a better understanding of the disease and the effects of treatment in order to improve the treatment regime and therapy. The patient care system according to the invention allows remote patient treatment monitoring for health professionals, provides information for decision making to HCP's, enables ease of administering medications for patients, provides easy health communication between HCPs and patients globally, and provides a safe, effective and economical patient care.

The computerized patient care system according to the invention may be referred to as an eHealth medical platform. Advantageously, the platform is secure and modular. The platform provides a set of applications to share information among care givers and patients in order to monitor and act upon a patient's condition, adherence to a dosage regimen and progress of a disease. The invention enables a care giver or a patient to monitor the adherence to a medical treatment regimen and to monitor the efficacy of a medical treatment regimen. The patient care system according to the invention has numerous benefits, including: supporting continuous care delivery with current and accurate information, engaging patients towards improving their lifestyle, health and quality of life, integrating care plans with relevant mobile user interface device, collecting and relying on real world evidence to keep improving healthcare, and generating data and insight that enable care improvements.

1. An interactive patient care system configured to provide care to a patient suffering from a chronic disease or condition, comprising a server system (6) configured to receive and transmit data via a communication network (16) to and from users including patients (15a) and health care professionals (15b), the server system comprising a processing unit (6e), a memory (6f), a patient database (6c) configured to store data related to the patient and a library database (6d) configured to store data related to predefined therapeutic interventions obtained on evidence-based care pathways, the server system further comprising an application server (6b) and a communication server (6a) including a web server software application for data transfer through the internet, wherein the application server (6b) comprises a patient care system software for chronic disease or condition management, configured to: i) select for a given patient at least one predefined therapeutic intervention from the library database based at least on the data related to the patient stored in the patient database, and ii) generate a personalized care plan based at least on the selected predefined therapeutic intervention, wherein the patient care system software comprises patient therapy services (40) comprising health monitoring components (40c) configured to monitor health parameters and/or treatment adherence parameters of the patient, and wherein said therapy services are configured to provide health care assistance to the patient based on said personalized care plan.

2. Patient care system according to claim 1, wherein said personalized care plan is generated based on one or more additional parameters selected from the group consisting of patient's goals, patient's preferences, patient's schedules, input provided by the patient and patient's historical monitored data.

3. Patient care system according to claim 1 or 2, wherein the patient care system software comprises a patient information management program with includes a reports component configured to generate reports, in the form of tables, charts, lists, diagrams or graphical representations based on information selected from drug administration history, adherence data, patient outcome reports, patient health reports, patient physiological data reports, medical device settings, treatment regimen data, and any combination of aforesaid information.

4. Patient care system according to claim 3, wherein the reports component are configured to form composite reports for simultaneous display on a user interface device (UID) display, including composite adherence and patient outcome reports to facilitate evaluation of the effects of non-adherence to the treatment regimen or the efficacy of the medical treatment.

5. Patient care system according to any preceding claims, wherein said patient therapy services (40) include tests (40a) accessible online by patients, said monitor components configured to capture results of tests, such as vision and walking tests.

6. Patient care system according to any preceding claim, wherein said patient therapy services include training exercises (40b) accessible online by patients, including training exercises selected from cognition training and mobility training.

7. Patient care system according to claim 5 or 6, wherein said patient therapy services may further be configured to receive data from sensors relating to physiological measurements of the patient's measured physiological data captured automatically by sensors and transmitted to the server system via the patient's user interface device and/or medical device and/or by the sensing or training device, physiological data selected from body temperature, blood pressure, pulse rate, electroencephalography measurements, electrocardiography measurements, breathing sensor measurements, blood sugar sensor measurements.

8. Patient care system according to any preceding claim, wherein the patient therapy services comprise web-based software which resides on the application server.

9. Patient care system according to any preceding claim, wherein the patient therapy services comprise software residing on a patient's user interface device.

10. Patient care system according to any preceding claim, wherein the communication server further comprises a remote service data upload software application configured for wireless telecommunication technology (WTT) data transfer.

11. Patient care system according to claim 10, further comprising a client side software application installable on a personal computer or on a mobile user interface device (UID) (10), such as a phone or computer tablet, configured to upload medical device usage data to a patient information management program application via the WTT remote service data upload application on the server system.

12. Patient care system according to any preceding claims, wherein the patient care system software further comprises prioritization and care coordination management (50) configured to allow health care professional to review monitored patients when said monitored heath parameters present deviations or anomalies that exceed a predetermined threshold value.

13. Patient care system according to claim 12, wherein said prioritization and care coordination components are configured to generate different levels of signals based on the severity of said deviations or anomalies, wherein said signal level is used to prioritize patients for care assistance, said prioritization and care services being optionally connected to a call centre.

14. Patient care system according to any preceding claim, wherein the patient care system software further comprises a notification services software component (30b) configured to transmit notifications by email, SMS (Short Message Service) and/or an application operating on a mobile device to patients and optionally other users of the system.

15. Patient care system according to claim 14, wherein said notifications are selected from the group consisting of encouragement, tip, request for patient to complete an activity and questions to be answered by the patient.

16. Patient care system according to the preceding claim, wherein the notification services software component is configured to send messages to the patients based on one or more types of parameters selected from the group consisting of monitored heath parameters, monitored treatment adherence parameters and target parameters.

17. Patient care system according to claim 16, wherein said target parameters are set in the personalized care plan of the patient.

18. A method of monitoring treatment and providing care to a patient suffering from a chronic disease or condition, comprising :
providing a computerized patient care system comprising a server system configured to receive and transmit data via a communications network to and from users including patients and health care professionals, the server system comprising a patient database configured to store data related to the patient, a library database configured to store data related to predefined therapeutic interventions obtained on evidence-based care pathway, an application server including a patient care system software for chronic disease or condition management, and a communication server for data transfer through the communications network,
processing and storing data related to predefined therapeutic interventions obtained on evidence-based care pathway on the server system,
processing and storing data related to patient care on the server system,
selecting at least one predefined therapeutic intervention based at least on the data related to the patient care,
generating a care plan template based at least on the selected predefined therapeutic intervention;
generating a personalized care plan for the patient based at least on the care plan template, and
providing patient therapy services to assist the patient to follow his personalized care plan.

19. Method according to claim 18, wherein the patient therapy services comprise one or more wellness services selected from the group consisting of coaching services, encouragement for behaviour change and online exercises.

20. Method according to claim 18 or 19, wherein the patient therapy services comprise e-Health therapies which includes medical devices designed to assess or contribute to the care of a specific chronic disease or condition.

21. Method according to claim 20, further comprising providing a client side software application installable on a personal computer or on a mobile user interface device (UID), such as a phone or computer tablet, configured to upload medical device usage data to a patient information management program application via a WTT remote service data upload application on the server system.

22. Method according to any one of claims 18 to 21, wherein the method further comprises providing the patient care system with a notification services software component (30b) configured to transmit notifications by email, SMS (Short Message Service) and/or an application operating on a mobile device to patients and optionally other users of the system.

23. Method according to claim 22, wherein said notifications are selected from the group consisting of encouragement, tip, request for patient to complete an activity and questions to be answered by the patient.

24. Method according to the preceding claim, wherein the notification services software component is configured to send messages to the patients based on one or more types of parameters selected from the group consisting of monitored heath parameters, monitored treatment adherence parameters and target parameters.

25. Method according to claim 24, wherein said target parameters are set in the personalized care plan of the patient.
